(19) United States
(12) Patent Application Publication
(10) Pub. No.: US 2003/0166301 A1
(43) Pub. Date: Sep. 4, 2003

(54) Multiple binding moiety chromatography device, methods of using and methods of making same

(76) Inventors: Irving W. Wainer, Washington, DC (US); Ruin Moaddel, Germantown, MD (US); Jean-Francois Cloix, Mereville (FR); Gozen Ertem, Washington, DC (US)

Correspondence Address: Supervisor, Patent Prosecution Services, PIPER RUDNICK LLP, 1200 Nineteenth Street, N.W., Washington, DC, 20036-2412, US

(21) Appl. No.: 10/315,056

(22) Filed: Dec. 10, 2002

Related U.S. Application Data
(60) Provisional application No. 60/337,172, filed on Dec. 10, 2001.

(51) Int. Cl. G01N 33/543

(52) U.S. Cl. 436/518

(57) ABSTRACT

The present invention is directed to a chromatography device with a stationary phase containing multiple binding moieties. The binding moieties are first solubilized and then immobilized on a stationary phase to create a multiple binding moieties phase for use in a chromatography device. In an alternative to the stationary phase embodiment, a single binding moiety can be directly bonded covalently to a support within the chromatography column. Combinations of constructions involving stationary phase immobilization and direct covalent bonding can also be employed. The multiple binding moiety chromatography devices are useful in investigating interactions among different binding moieties in pharmacological studies and in drug discovery.

This application takes priority from Provisional Application No. 60/337,172, filed Dec. 10, 2001. The entirety of which, and all references cited herein, are incorporated by reference for all purposes.

### BACKGROUND OF THE INVENTION

1. Field of the Invention

The present invention relates generally to the development of a stationary phase, and a chromatography device containing this stationary phase. The stationary phase is particularly formulated for use in the chromatography device and contains multiple species of binding moiety sites for interaction with a molecule such as a drug, or drug candidate. The binding moiety may be any protein, such as a receptor, an enzyme or a transport protein. Typical sources for the binding moiety in the invention include animal tissue, expressed cell lines or commercially synthesized proteins.

More particularly, the invention is concerned with the application of high performance liquid chromatographic systems containing multiple protein moiety species as binding sites. These systems can be employed in such diverse fields as organic synthesis, biochemistry and pharmacology.

According to one aspect of the invention, the binding moieties in the formulated stationary phase are immobilized in the stationary phase without covalent binding to the chromatography column, but instead are enveloped in the stationary phase. The chromatography devices according to the invention can be used in displacement chromatography, frontal or zonal chromatography and other forms of chromatography to identify lead candidate molecules having a similar specific binding affinity as compared with one or more markers molecules. A marker molecule, by definition, has a known specific binding affinity for a distinct species of binding moiety in the chromatography device.

In one method according to the invention, the chromatographic system is used to measure displacement of a marker molecule by the sample molecule in order to determine from the eluate an assessment of the sample molecule as a candidate for drug development.

The chromatography device according to the invention allows simultaneous assessment of the binding affinities of the sample against multiple species of binding moiety. In another aspect of the invention, the presence of multiple binding moieties allows assessment of the interaction of the multiple binding moieties in response to introduction of one or more samples.

Another aspect of the invention is a chromatography device that can be used in displacement chromatography, frontal or zonal chromatography and other forms of chromatography wherein the binding moiety is covalently bound directly to a support within a column, optionally without the presence or reliance upon a stationary phase for binding moiety immobilization.

The present invention is also generally concerned with chromatographic systems wherein binding moieties may be drawn from among different species of one type of one protein moiety, such as two receptor species. Protein moiety types are differentiated by function and can include functionality classifications based on cellular activity such as receptors, cell membrane transporters or channel proteins, or enzymes.

Alternatively, a chromatography device according to the invention can be made using separate binding moiety species from among different functional types of protein moieties, such as a combination of a receptor binding moiety and an enzyme binding moiety, for analysis of their interaction upon a sample.

One aspect of this invention relates to a method of securing a single species of protein as the binding moiety. In this embodiment, a protein is covalently secured through a linker or spacer directly to a support within a chromatography column. The invention relates to chromatography devices using this construction, either for a single species of protein, or in a combination involving more than one species of protein or protein functional type.

In another aspect of the invention, the present invention demonstrates that multiple binding moiety species, or protein functional types such as receptors, transporters or enzymes, can be solubilized and then immobilized on an immobilized artificial membrane (IAM) liquid chromatographic stationary phase.

The invention also relates to chromatography devices having at least one covalently secured binding moiety in combination with at least one binding moiety immobilized in a stationary phase. Binding moieties in a stationary phase can be either secured directly to a support within the chromatography column by direct covalent binding, or can be enveloped in the stationary phase without any covalent attachment to a support within the chromatography column. The multiple binding moiety chromatography device of the invention can be formed using a stationary phase to hold a binding moiety in a chromatography column, or by directly securing a protein moiety to a support in the column, or by a combination of these methods.

2. Discussion of the Background

In the classical approach to drug discovery, compounds of unknown function or effect were given to animals and the pharmacological and toxicological reactions were determined. Subsequently, pharmacological targets were identified such as, but not limited to, receptors, enzymes and transport proteins. This led to the development of activity and binding assays using solubilized or immobilized targets. These assays were used to screen test compounds for biological activity. In this approach, a specific interaction between the target and the test compound was an indication that the compound could have in vivo pharmacological activity.

The combinatorial synthesis of chemical libraries has created an enormous pool of possible new drug candidates. The therapeutic and toxic effects of drugs and drug candidates are often controlled by the interaction these drug candidates have with biopolymers such as receptors and enzymes. Synthetic method capabilities, including phage display preparations, have outstripped the ability to determine the corresponding biological activity for each of these interactions. An initial step in the resolution of this problem has been the development of microtiter plates which contain immobilized receptors and antibodies. The use of these plates can rapidly reduce the number of possible candidates in a combinatorial pool from thousands to hundreds. However, assignment of relative activity within the reduced pool of compounds remains a slow and repetitive process.

The relationship between basic pharmacological processes and liquid chromatography studies have been emphasized by the inclusion of biomolecules as active components of chromatographic systems. A wide variety of immobilized biopolymer-based liquid chromatography stationary phases have been developed using a single species of protein, enzyme, antibody or liposome.

The therapeutic and toxic effects of drugs are governed by the interactions of the drug molecule with natural binding moieties such as receptors, cell membrane transporters and enzymes. The binding moiety-drug interaction define a drugs's pharmacological fate. As such, there have been interdisciplinary efforts amongst fields such as medicine, pharmacology and biochemistry to develop methods for identification or characterization of these reactions.

The acceleration in drug discovery activity, with the disproportionately higher increase in drug development cost per candidate has created a need for an improved means of screening for candidate drugs. These needs include not merely a reduction in testing time, but also in the quality of data that results.

Modern drug discovery is based upon the identification and validation of disease-related targets. This approach has been coupled to the production of enormous pools of possible new drug candidates created by combinatorial chemical synthesis. Classical binding assays cannot efficiently screen the combinatorial pools as synthetic capabilities have outstripped the ability to determine corresponding biological activity.

Previous work toward overcoming this problem was the development of high throughput screening techniques primarily based upon microtiter plates containing an immobilized target. However, high throughput screening with microtiter plate technology rapidly generates a large amount of data, but the data has limited information content. There is a pressing need for multiple screens and complex data management.

In drug development “Lead Optimization” is the process of going from an active compound to a new drug candidate for clinical testing. It involves the determination of how much of the compound will enter the body (adsorption {A}), where the compound will go once it is in the body (distribution {D}), what the body will do to the compound and the consequences of any metabolic transformations (metabolism {M}), how the body will get rid of the compound (excretion {E}), and the toxicological effect the drug will have as it enters and is metabolized in a subject (toxicology {T}). This process is identified as the ADMET stage of drug development.

New drug discovery programs often identify hundreds of compounds that have activity at a disease-related target. The ADMET stage is used to determine which compounds will have the best chance of becoming a drug. Poor performance in one or more of the ADMET studies will often eliminate the compound from the development program. The ADMET screen is done primarily for economic reasons as the next stages in the drug development program will involve in vivo animal studies, which consume a great deal of time and resources. Thus, the ADMET program is designed to identify a limited number of compounds for further testing and, thereby, optimize the chances of success.

Time is the greatest single expense in drug discovery and development. The ADMET stage still requires large amounts of time even in today's increasingly automated and high-throughput oriented discovery and development environment. Pharmacological evaluation is a rate-limiting step in drug discovery. Although high throughput testing with microtiter plates has partially alleviated this problem, there are still many possibilities for improvement.

The different binding moieties with which a drug candidate will interact in these phases can generally be classified according to the in vivo function the binding moiety has within the cell and this typically includes receptors, enzymes and transport proteins.

RECEPTORS—Drugs can affect localized regions in vivo. For instance, drugs active in the central nervous system (CNS) exert their pharmacological activities by affecting a number of CNS receptors. These receptors include a variety of neurotransmitter receptors classified as the ion channel receptor superfamily. When activated, these receptors transmit a signal by altering the cell membrane potential or ionic composition.

The ion channel receptor superfamily is composed of three groups of receptors: the nicotinic, excitatory amino acid, and ATP purinergic receptors. In turn, the nicotinic receptor family is further subdivided into subfamilies of nicotinic (NCT), GABAa, GABAc, Glycine receptors, 5-HT3 (serotonin) receptors. The same is true for the excitatory amino acid receptor family that is composed of glutamate, N-methyl D-aspartate (NMDA), AMPA, and kainate receptors. While the general biochemical mechanism is the same throughout the ion channel superfamily, there are dramatic differences in pharmacology, ion selectivity, and response to allosteric modulators between and within the families and subfamilies. Goodman and Gilman 's The Pharmacological Basic of Therapeutics Ninth Edition, McGraw Hill Publishers, New York, pp. 32-33 (1996).

Although there are great differences in the ion channel superfamily, there are also significant overlaps. As such, a drug specifically designed for one receptor subtype may also elicit a response at another. For example, risperidone, an anti-psychotic agent, binds to both the dopamine (D₂) and the 5-hydroxytryptamine receptors. Norman et al., J. Med. Chem., 39:(1996)1172-1188. While buspirone binds to both α₁-adrenergic and 5-hydroxytryptamine receptors. Lopez-Rodriquez et al., Bioorganic and Med. Chem. Letters, 6(6):(1996) 689-694. At the present time, it is difficult to determine the effect of a drug or a drug candidate on the individual members of a multiple receptor system. Indeed, the extreme complexity of such systems makes it hard to rationally design specific tools to directly mimic multiple receptor biological systems. However, the development of receptor-based liquid chromatographic stationary phases has opened up the possibility for the development of on-line multiple-receptor screens.

Previous studies have reported the immobilization of two of the members of the NCT receptor family, the α4/β2 and α3/β4 NCT receptors, to create NCT receptor-based stationary phases (NR-SPs). Zhang et al., Anal. Biochem., 264:(1998) 22-25; Zhang et al., J. Chromatogr. B, 724:(1999) 65-72. The NR-SPs were used in liquid chromatographic studies employing known NCT receptor ligands. The order and magnitude of the binding affinities obtained in these studies were the same as those obtained with standard binding assays. Thus, the results imply that the NR-SPs can be used as an on-line screen for NCT receptor ligands.

Additionally, in the previous studies, the α3/β4 NCT receptor-subtype was obtained from a transfected cell line expressing this receptor while the α4/β2 NCT receptor-subtype was prepared from rat forebrain tissue. The resulting NCT-SPs could not only be used to assess ligand binding affinities, they could also be used to determine differences between the two receptor subtypes.

Of course, the same challenges in assessing multiple binding moiety interaction presented with respect to NR-SPs is also present with respect to other types of receptors. In addition, many receptors have no known ligand. Multiple binding moiety assessment can generate data useful in determining the function of these uncharacterized receptors.

ENZYMES—Enzymatic transformations are extensively used in organic chemical synthesis and in the metabolism and pharmacological activity of drugs. A wide variety of enzymes are used in these processes. In recent years, there have been significant developments in the study of the basis of enzyme-drug interactions. The understanding of how enzymes react with drugs and bring about chemical changes in vivo is a key factor for the determination of drug pharmacodynamics and pharmacokinetics, and is also important in the development of new therapeutic agents.

While a number of useful methods have been utilized in the production of immobilized enzyme reactors (IMERs), the most popular are non-covalent entrapment and covalent attachment. Non-covalent entrapment has been achieved using the immobilized artificial membrane stationary phase (IAM-SP). Pidegon, C. (1990), Enzyme Microb. Technol. 12, 149-157. The IAM-SP is derived from the covalent immobilization of 1-myristoyl-2-[(13-carboxyl)tridecanoyl)]-sn-3-glycerophosphocholine on aminopropyl silica, and resembles one-half of a cellular membrane. In the IAM-SP, the phosphatidylcholine headgroups form the surface of the support and the hydrocarbon side chains produce a hydrophobic interface that extends from the charged headgroup to the surface of the silica. With the IAM interphase, enzymes are embedded within the interphase surroundings.

Covalent attachment of enzymes to chromatographic stationary phases has been accomplished using Glutaraldehyde-P. This packing is a wide pore silica that has been covalently clad with a hydrophilic polymer, polyethleneimine. Narayanan et al., Anal. Biochem. (1990) 188, 278-284. Another method for the covalent immobilization of enzymes on a chromatographic support has been described by Zhang, et al. Anal. Biochem. (2001) 299, 173-182. In this approach, membranes containing the target enzyme are biotinylated and adsorbed onto beads containing immobilized streptavidin. This procedure has been used to immobilize recombinant human N-acetylglucosaminyltransferase V.

TRANSPORT PROTEINS—P-glycoprotein (Pgp) is a 170 kDa cell membrane protein, and a member of the ATP binding cassette (ABC) superfamily of transport proteins. This superfamily includes the multi-drug resistance-associated protein (MRP1), the canalicular multi-specific anionic transporter (cMOAT, or MRP2), the breast cancer resistance protein (BCRP), and the cystic fibrosis transmembrane conductance regulator (CFTR). Pgp is an efflux drug transporter whose substrates include anticancer drugs such as the anthracycline antibiotics and vinca alkaloids, steroids, verapamil, peptides and quinolines.

This broad substrate specificity has not been definitively explained and represents a central question of Pgp biology. Pgp presents different possible models to explain Pgp activity including both as a membrane vacuum cleaner mechanism in which Pgp binds its substrates from the inner leaflet of the plasma membrane and releases it into the extracellular fluid. Pgp activity has also been described as a flipase that transports the substrate from the inner to outer leaflet of the plasma membrane.

The number of binding sites on the Pgp molecule has not been determined and there is evidence for the existence of multiple binding sites as some substrates bind to Pgp in a mutually non-competitive manner. Other data suggest synergistic activity or the differential effect based on the presence of multiple binding sites.

Previous studies with an immobilized P-glycoprotein transporter in a stationary phase have demonstrated that allosteric interactions can also be detected using displacement chromatography through changes in elution volume. Zhang et al., J. Chrom. B, 739:(2000) 33-37. In this system, cooperative allosteric interactions produced increased elution volumes while anti-cooperative allosteric interactions eliminated all of the observed specific retention. Lu, et al., Pharm. Res., 18:(2001) 1327-1330.

Thus with respect to receptors, enzymes or transport binding moieties, as discussed above, the immobilization of a single binding moiety species in a stationary phase, and chromatography devices made using such stationary phases are known. See Wainer et al., U.S. Pat. No. 6,139,735. However, chromatography devices having multiple species of binding moieties in one stationary phase are not known in the art, as also are stationary phases formulated to contain multiple binding moieties.

Chromatography devices wherein a single species of binding moiety is enveloped in a stationary phase are known. See Wainer et al., U.S. Pat. No. 6,139,735. Currently, a chromatography device in which the binding moiety is covalently bound directly to a support within the column have not been reported. Also, chromatography devices in which multiple binding moieties are either covalently attached to the column wall, or are present in combination within a stationary phase, optionally containing non-covalently bound binding moieties are also not known.

### SUMMARY OF THE INVENTION

The invention relates to stationary phases formulated to having multiple species of distinct binding moieties in one stationary phase. It is a further object of the invention to solubilize and immobilize multiple protein moiety binding sites on an IAM stationary phase so as to make a multiple binding moiety-stationary phase. The invention also relates to a chromatography device containing multiple species of binding moieties in one stationary phase or in one chromatography device having multiple stationary phases having one or more distinct species of binding moiety.

The invention is also directed to making chromatography devices wherein a binding moiety is covalently bound directly to a support within a column. The invention further relates to chromatography devices in which multiple species of binding moieties are either covalently attached to a support within a column, and optionally are present in combination with a stationary phase, optionally containing non-covalently bound binding moieties in that stationary phase.

It is yet another object of the invention to utilize in methods of displacement chromatography, frontal or zonal chromatography and other forms of chromatography these multiple binding moiety chromatography devices wherein the moieties are directly bound to a support within the chromatography column, optionally in combination with a stationary phase, which optionally contains additional binding moieties which are not covalently attached to any support within the chromatography column.

It is another object of the invention to use multiple binding moiety stationary phase containing columns to investigate single interactions between ligands or molecules and receptors, enzymes, or transport proteins and identify the differences in binding among various receptors, enzymes, or transport proteins.

It is a further object of the invention to identify new drug candidates from a library or pool of potential compounds using the chromatography devices of the invention. More particularly, the object of the invention includes utilizing the chromatography devices of the invention in determining how much of a compound will enter the body (i.e., adsorption), where a compound will go once it is in the body (i.e., distribution), what the body will do to a compound and the consequences of any metabolic transformations (i.e., metabolism), how the body will eliminate a compound (i.e., excretion), and the toxicological effect a compound will have as it enters and is metabolized in a subject (i.e., toxicology).

It is yet another object of the invention to isolate known and unknown compounds from a complex biological matrix using the chromatography devices of the invention.

It is yet another object of the invention to utilize the chromatography devices of the invention to identify ligands or other molecules with some binding affinity with uncharacterized binding moieties, including, but not limited orphan receptors, orphan enzymes and orphan transport proteins.

### BRIEF DESCRIPTION OF THE DRAWINGS

FIG. 1 is a graphical illustration of the elution profiles of (³H)-EB on a multiple receptor stationary phase (0.5×1.7 cm) and 60 pM (³H)-EB with the presence of 1 μM NCT in the mobile phase.

FIGS. 2A and 2B are graphical illustrations where the plot of elution volume (percent maximum response) as a function of MK-801 concentration was used to determine the K_{d} of MK-801 for the NMDA receptor. The K_{d} obtained by this method was 0.6 nM (FIG. 2A) and 1.2 nM (FIG. 2B).

FIGS. 3A and 3B are graphical illustrations of the frontal chromatography on a co-immobilized multiple receptor stationary phase. FIG. 3A illustrates the elution profile of a 60 pM solution of the NCT receptor ligand (³H)-EB* alone. FIG. 3B illustrates the GABA_{A} ligand FTZ added to the mobile phase at a 1 μM concentration.

FIGS. 4A and 4B are graphical illustrations of the frontal chromatography on the co-immobilized MR-SP where FIG. 4A illustrates the elution profile of a 25 pM solution of the GABA_{A} ligand (³H)-FTZ* alone. FIG. 4B illustrates receptor ligand (−)-NCT added to the mobile phase at a 1 μM concentration.

FIG. 5 is a chromatogram showing a run illustrating the elution profile of 0.5 nM 3H-Vinblastin displaced with 125 nM cold vinblastin (right) and 500 nM cold vinblastin (left) at 50 ul/min with 10 mM Amm Acetate pH 7.4 as mobile phase. Run on an open tubular PGP column (PGP-OTB).

### DETAILED DESCRIPTION OF THE INVENTION

Definitions

RECEPTOR—In general, a receptor is any protein (ie. membrane-bound or membrane enclosed molecule, water soluble or cytosolic) that binds to, or responds to something more mobile (i.e., the ligand), with some level of specificity. The level of specificity can be high, selective or low. Low specificity binding is often characterized as “dirty” or “promiscuous.” Examples include acetylcholine receptor, adenosine receptors, adrenergic receptors, adrenomedullin receptor, Ah receptor, amino acid receptors, AMPA (α-Amino-3-hydroxy-5-methyl-4-isoxazole propionic acid) receptor, ANP receptor, androgen receptor, baroreceptor, calcitonin gene related peptide receptor, cannabinoid receptors, chemokine receptors, chemoreceptor, Con A receptors, death receptors, EGF receptor, endothelin receptor, estrogen receptor, Fc receptors, fibroblast growth factor receptor, G-protein-coupled receptor, GABA (gamma aminobutyric acid) receptor, glutamate receptor, glycine receptor, growth factor receptor bound protein 2, glutamate receptor interacting protein, imidazoline receptors, IL-1 receptor associated kinase, insulin receptor substrate-1, immunoreceptor tyrosine-based activation motif, killer cell inhibitory receptor, killer cell immunoglobulin-like receptor, leptin receptor, low density lipoprotein receptor, muscarinic acetylcholine receptor, NCT receptors, α3/β4 NCT receptor-subtype, α4/β2 NCT receptor-subtype, nuclear receptor corepressor, nicotinic acetylcholine receptor, NMDA (N-methyl-D-Aspartate) receptor, nuclear receptor, opioid receptors, peptide neurotransmitter receptor, photoreceptors, peroxisome proliferator-activated receptors, presynaptic receptors, protease-activated receptors, purinergic receptors, receptors for activated C Kinase, receptor tyrosine kinases, scavenger receptors, serpentine receptors, signal recognition particle-receptor, steroid receptor, sulphonylurea receptors; T-cell receptor, TNF receptor, and vanilloid receptor-1, thyroid hormone receptors, retinoic acid receptor, progesterone receptor, glucocorticoid receptors, nuclear receptors and others including proteins that can also be classified channel proteins such as, ligand gated ion channels, voltage gated ion channels, potassium channel, calcium channel. This definition also includes orphan receptors.

ENZYME—An enzyme is any protein, natural or synthetic, that can catalyze one, and usually only one, specific biochemical reaction. Six functional types of enzymes are recognized which catalyze the following reactions: (1) redox (oxidoreductases), (2) transfer of specific radicals to groups (transferases), (3) hydrolysis (proteolytic), (4) removal from or addition to the substrate of specific chemical groups (lysases), (5) isomerization (isomerases), and (6) combination or binding together of substrate units (ligases). Specific examples include: abenzyme, angiotensin converting enzyme, apoenzyme, exoenzyme C3, catalytic antibody (i.e., abenzyme), coenzymes, coenzyme A, coenzyme M, coenzyme Q, ectoenzyme, endothelin converting enzyme, exoenzyme, holoenzyme, hydrolytic enzymes, interleukin-1 converting enzyme, isoenzymes, lysosomal enzymes, metalloenzyme, modification enzyme, N-acetylglucosaminyltransferase V, pro-enzyme, proteolytic enzyme, Q enzyme, restriction endonucleases or restriction enzymes, and coenzyme Q. This definition also includes orphan enzymes.

Most known enzymes are assigned an EC number by the Enzyme Commission and are listed in the ENZYME database at http://us.expasy.org/, the entire repository of which is incorporated by reference as of the filing date of this application. EC numbers are assigned primarily based on the recommendations of the Nomenclature Committee of the International Union of Biochemistry and Molecular Biology (IUBMB). The ENZYME database contains the physical and functional data and known characteristics for each type of characterized enzyme for which an EC (Enzyme Commission) number has been provided.

TRANSPORT PROTEIN—Transport proteins are any of the class of proteins involved in the transfer of a substance from one side of a plasma membrane to the other. The transport can be in a specific direction and can be at a rate faster than diffusion alone. Transport proteins that merely facilitate the diffusion of molecules or ions across a lipid membrane by forming a lipid pore are also called channel proteins. Also involved in transport are channel proteins. Specific examples of transport proteins include P-glycoprotein, and any of a class of protein that have been identified with active transport of a particular substance. These proteins include channel protein types such as A-channel, calcium channel, channel-forming ionophore, chloride channel, delayed rectifier channels, gated ion channel, G-protein-gated inward rectifying potassium channels, ion channel, L-type channels, ligand-gated ion channel, M-channels, N-type channels, P-type channels, potassium channel, Q-type channels, R-type channels, sodium channel, T-type channels, voltage-gated ion channel, and voltage-sensitive calcium channels. This definition also includes orphan transport proteins.

CYTOSOLIC PROTEIN—A protein, when fully developed in vivo, resides and functions in the cellular cytosol, or in the extracellular space.

MEMBRANE PROTEIN—A protein, when fully developed in vivo, has regions of the protein permanently attached to a membrane, or inserted into a membrane.

PERIPHERAL MEMBRANE PROTEIN—A protein, when fully developed in vivo, that is bound to the surface of the membrane and not integrated into the hydrophobic region.

TRANSMEMBRANE PROTEIN—A membrane protein having a protein subunit in which the polypeptide chain is exposed on both sides of the membrane, or having different subunits of a protein complex that are exposed at opposite surfaces of the membrane.

BINDING MOIETY—A peptide or nucleotide containing moiety having a known binding affinity for at least one marker molecule. The moiety can be a protein, a polypeptide, a protein fragment (such as an antibody fragment) or one or more subunit(s) of any protein. A typical example of a binding moiety would be an enzyme, a receptor or a transport protein. It can also be a carrier protein such as albumin or an antibody. The binding moiety can also be, or include, a sequence of DNA or RNA.

MARKER MOLECULE—Any compound having a known binding affinity for a binding moiety.

DRUG DISCOVERY INVOLVING RECEPTORS—In addition to the combinatorial synthesis of large chemical libraries, genomic research has also led to the identification of thousands of genes representing potential disease-related targets. The expression of these genes has led to the identification of thousands of proteins of unknown function, often referred to as orphan receptors.

For example, the G-protein-coupled receptor (GPCR) family contains many orphan receptor species. The GPCR family is a broad ranging collection of membrane receptors that play a vital role in biological and pharmacological processes. Generally more than 50% of the current therapeutic agents are directed to known GPCR targets. Genomic studies indicate that the human genome encodes for more than one thousand GPCRs, but only about half have been identified. The rest are “orphan receptors.”

Orphan receptors are receptor proteins having no identified cellular function and can be the source of new disease-related targets and, consequently, create the ability to find new treatments for a wide variety of diseases. Microtiter plate-based high throughput screens have limited application in this search for new drugs. These screens require the identification of at least one compound that binds to the target, and, for orphan receptors this is generally not known. Microtiter plate technology is reaching its limits in addressing this research problem. The pharmaceutical industry needs high throughput screens that have high information content and that can be used with known disease-related targets and with orphan receptors.

According to the invention, the disease-related target or orphan receptor alone is covalently immobilized on a solid support, or combined with another target binding moiety in a stationary phase on a support, and the support is packed into a small column and the column placed in a flowing chromatographic system. The test chemicals are passed through the column, over the immobilized target, and the time that it takes for the compounds to pass from the beginning of the column to its end is directly related to the strength of interaction between the target and the compound, i.e. the binding affinity of the ligand-receptor complex. Using this method, complex chemical and biological mixtures can be rapidly sorted between compounds that interact and do not interact with the disease-related target. At the same time, the compounds that bind to the target are themselves rapidly sorted between low, medium and high affinity binders. Thus, the method quickly provides a large amount of data with high information content.

In the case of orphan receptors, membranes from cells expressing the orphan receptor are used to create an experimental column and membranes from cells that do not express the orphan receptor are used to create a control column. Test compound(s) can be simultaneously injected onto both columns. If the test compound(s) take longer to pass through the experimental than through the control, this will indicate that the compound has an affinity for the orphan receptor. In this way, compounds can be rapidly screened for their ability to interact with an orphan receptor. In addition, the orphan receptor target can be combined with another target binding moiety on one column to assess interaction between the orphan receptor and another binding moiety.

By extension, this analysis can be extended to other binding moieties that are “orphans” in that they are not fully characterized, such orphan enzymes and orphan transport proteins.

DRUG DISCOVERY INVOLVING ENZYMES—The in vivo fate of a compound is often determined by its interaction with endogenous enzymes, which transform the compound into a variety of new compounds, metabolites. The clinical effect of these changes varies from compound to compound, the metabolite may have no therapeutic activity, it may have more therapeutic activity, or it may be toxic. At the present time, the metabolic fate (the M of the ADMET program) cannot be predicted and must be experimentally determined.

For instance, the cytochrome P450 family of enzymes is recognized as the primary mediator of drug metabolism. Current use of microtiter plate technology to determine which member(s) of the cytochrome P450 family play a role in the metabolic conversion of lead drug candidates. However, this technology cannot be readily used to identify the structure of the metabolite(s) or the effect of the conversion on the efficacy and toxicity of the lead drug candidate. The chromatographic devices according to the invention allow the combination of an enzyme target such as a cytochrome p450 enzyme, with another binding moiety to assess their interaction.

DRUG DISCOVERY INVOLVING TRANSPORT PROTEINS—Recent pharmacological studies have identified a family of proteins that play a role in the transportation of drugs across cellular membranes, the ABC transporter family. These transporters primarily affect the adsorption and excretion (A, E) of drugs, but are also involved in all other stages of the ADMET program. Therefore, the interaction of compounds with these transporter proteins is an important component of the ADMET program.

Current studies involving transport proteins require the use of cell lines. These studies are time consuming, expensive and often inaccurate. The human genome suggests that there are over one hundred members of the ABC transporter protein family the majority of these have not been identified or characterized (i.e., “orphan” transport proteins). Thus, the chromatographic devices according to the invention can be used to enhance drug discovery in the A and E components of the ADMET program, as well as characterize as yet unknown transport proteins.

DRUG DISCOVERY INVOLVING ALBUMIN AND OTHER CARRIER PROTEINS—The distribution of a drug is often dependent on its binding to endogenous proteins. A key component is the carrier proteins found in the blood, of which, serum albumin is the most prominent. In some cases, more than 99% of a drug that reaches the blood stream will be bound to serum albumin. To what extent and how a drug is bound to serum albumin is an important issue in drug development and a question posed by the U.S. Food and Drug Administration as part of the drug approval process.

Ultrafiltration and equilibrium dialysis are the two standard techniques used to determine the extent of binding to serum albumin. However, when the binding exceeds a certain extent these methods become inexact due to non-specific binding. There is a need for determining more specific binding to albumin and other carrier proteins. In addition, there is a need for testing specific binding of serum albumin from a variety of species including human, rat, mouse, pig and dog, and other animals used in laboratory testing.

MULTIPLE BINDING MOIETIES IN STATIONARY PHASE—Although the following demonstrates multiple receptors as the multiple binding moieties in a stationary phase, the invention is in no way limited to multiple receptors as the binding moieties. The invention may also be practiced with other binding moieties such as enzymes or transport proteins.

In previous studies, NCT receptors were isolated from rat brain tissues and immobilized on the IAM liquid chromatographic support. The resulting NCT receptor-stationary phase contained active NCT receptors that closely resembled the activity of the α4/β2 NCT receptor subtype isolated from rat brain tissues. Zhang et al., J. Chromatogr. B, 724:(1999) 65-72; Anderson et al., J. Pharmacol. Exp. Ther., 273(3):(1995) 1434-41.

The ability to use rat forebrain tissue to prepare a functioning NCT-SP raised the possibility of developing a liquid stationary phase containing more than one functioning receptor, i.e., a multiple-receptor stationary phase (MR-SP). In this regard, the present inventors pursued the development of a MR-SP containing different members of the LGIC superfamily obtained from rat forebrain tissue. In particular, the solubilized tissue was immobilized on an IAM liquid chromatographic stationary phase using previously described techniques (see Zhang et al., J. Chromatogr. B, 724:(1999) 65-72), and binding affinities were obtained using frontal chromatography techniques. On-line competitive binding experiments were performed using ligands for the NMDA receptor (MK-801), NCT receptor (epibatidine) and GABA_{A} receptor (flunitrazepam) as the marker ligands and NMDA, epibatidine or nicotine and diazepam as the respective displacer ligands. The results from these studies demonstrated that the NCT receptor, NMDA receptor, and GABA_{A} receptor were successfully immobilized on the same solid support and kept pharmacologically active and independent.

The data demonstrates that multiple-receptor liquid chromatographic stationary phases (MR-SPs) can be prepared and that these phases can readily yield a great amount of diversified, precise and reproducible data including interactions and overlap between the immobilized receptors. The MR-SPs also provide a novel approach to the rapid identification of pharmacologically important changes in receptor activity as well as the identification of CNS active substances in complex matrices.

In the present application, the previously described protocol was repeated with the same results relative to the immobilized NCT receptor. The data obtained demonstrates that the tissue solubilization and immobilization procedures are reproducible as are the affinities obtained on the resulting chromatographic phase.

In the initial studies, the IAM stationary phase was utilized because it is based upon the covalent attachment of a phospholipid monolayer onto the surface of a silica particle. Pidgeon et al. (eds), Applications of Enzyme Biotechnology, Plenum Press, New York, pp. 201-237 (1992). The form of the IAM stationary phase used in this study was composed of 12 μM silica particles with 300 Å pores. On electron micrographs, purified and membrane bound NCT receptors from Torpedo electric organ appear as a ring-like particle approximately 65 Å in diameter and 110 Å in length. Unwin, N., J. Mol. Biol., 229(4), (1993)1101-1124. Thus, it can be assumed that the solubilized NCT receptors were embedded in this monolayer, reflecting at least part of the receptor's transmembrane environment.

The co-immobilization of multiple receptors from rat brain tissue onto a stationary phase is novel, and the co-immobilized receptors identified were the NCT, GABA_{A} and NMDA receptors. These receptors, albeit pharmacologically independent, are part of the same superfamily of LGIC receptors. They are all composed of five separate polypeptide chains or subunits. Le Novere et al., Nucleic Acids Res., 27(1):(1999) 340-342. They differ only in the make-up of each subunit and are relatively similar in size. The subunits of NCT and GABA_{A} receptors are composed of a large extracellular N-terminal domain, with four transmembrane domains and an extracellular carboxy terminus. The NMDA receptor subunits are composed of a large extracellular N-terminal, three transmembrane domains, a P loop, and an intracellular carboxy terminus. Thus, it is presumed that the GABA_{A} and NMDA receptors would mimic the NCT receptor and also be embedded in the phospholipid monolayer of the IAM stationary phase.

In the present invention, the dialysis process produced the immobilization of the solubilized receptors on the IAM particles. As the detergent concentration decreased, the receptors were driven into the phospholipid monolayer. This process produced independently immobilized receptors throughout the IAM particles.

In displacement chromatography, the displacer ligand is placed in the mobile phase and the chromatographic phase is brought into contact with this phase. Once the system has reached equilibrium, the target ligand is then introduced into the system. Therefore, in the competitive displacement experiments undertaken in the present application, the MR-SP has been equilibrated with the specific GABA_{A} receptor ligand before introduction of the specific NCT receptor ligand or, conversely, equilibrated with the specific NCT receptor ligand before introduction of the specific GABA_{A} receptor ligand. The 1 μM concentrations were used in order to assure that the displacer ligand saturated the MR-SP. The fact that these experiments produced no observable changes in the elution volumes of the marker ligands reflects the specificity of the marker ligands used, as well as the independence of the immobilized receptors. An overlap in either of these factors would have been reflected in a change in the elution volume.

Having generally described this invention, a further understanding can be obtained by reference to certain specific examples which are provided herein for purposes of illustration only and are not intended to be limiting unless otherwise specified.

### EXAMPLES

Materials: (³H)-EB, (³H)-FTZ, (³H)-MK-801 were purchased from Amersham Life Science Products (Boston, Mass., USA). NMDA, (−)-NCT, benzamidine, NaCl, MgCl₂, CaCl₂, KCl, cholate, leupeptin, phenyl methyl sulfonyl fluoride (PMSF), EDTA, Trizma base, and Trizma-HCl were purchased from Sigma Chemical Co. (St. Louis, Mo., USA). Scintillation liquid (Ultima Flo-one) was purchased from the Georgetown University Chemical Stock room (Washington, D.C., USA). The chromatographic backbone (Immobilized Artificial Membrane PC Stationary Phase (IAM)) was obtained from Regis Chemical Co. (Morton Grove, Ill., USA). Rat brains were purchased from Pel-Freez Biologicals (Rogers, Ark., USA).

### Example 1

Preparation of Multiple Receptor Stationary Phase (MR-SP)

Solubilization of rat brain tissue: All of the tissue from four rat brains was homogenized in 30 ml of Tris-HCl buffer (50 mM, pH 7.4) containing 5 mM EDTA, 3 mM benzamidine and 0.2 mM PMSF for 3×20 seconds using a Polytron homogenizer (Brinkman Instruments, Westbury, N.Y., USA) at setting 6. The mixture was kept in an ice bath for 20 seconds between each homogenization step to prevent excessive heating of the tissue. The homogenized brain tissue was centrifuged for 10 minutes at 4° C. at 35,000×g, and the supernatant was discarded. The pellet was suspended in 10 ml of Tris-HCl buffer (50 mM, pH 7.4) containing 100 mM NaCl, 2 mM MgCl₂, 3 mM CaCl₂, 5 mM KCl, 2% sodium cholate, and 10 μg/ml leupeptin. The resulting mixture was stirred for 12 hours at 4° C. and centrifuged at 35,000×g.

Immobilization of solubilized receptors: The supernatant (receptor-cholate suspension) was mixed with 200 mg of dried IAM-PC packing material and stirred gently for 1 hour at 25° C., transferred into dialysis tubing, and dialyzed for 48 hours at 4° C. against 3×600 ml of Tris-HCl buffer (50 mM, pH 7.4) containing 5 mM EDTA, 100 mM NaCl, 0.1 mM CaCl₂ and 0.1 mM PMSF.

The resulting mixture was centrifuged for 3 minutes at 4° C. at 32,000×g, and the supernatant was discarded. The pellet (MR-SP) was washed with Tris-HCl buffer (50 mM, pH 7.4) and centrifuged. This process was repeated until the supernatant was clear. The MR-SP was then collected.

Determination of Biding Affinities Using Frontal Chromatography

Chromatographic procedures: The MR-SP (200 mg) was packed into a HR 5/2 glass column (Amersham Pharmacia Biotech, Uppsala, Sweden) to yield a 150 mm×5 mm (ID) chromatographic bed. The column was then connected to a P 1000 isocratic HPLC pump (Thermo Separations, San Jose, Calif., USA). The mobile phase consisted of Tris-HCl buffer (50 mM, pH 7.4) delivered at a flow rate 0.4 ml/min at room temperature. Detection of the (³H)-marker ligands was accomplished using an on-line scintillation detector (525 TR, Packard Instruments, Meriden, Conn., USA).

In the chromatographic studies, a 50 ml sample Superloop (Amersham Pharmacia Biotech) was used to apply a series of (³H)-marker ligand concentrations through the MR-SP column to obtain elution profiles showing front and plateau regions. The chromatographic data was summed up in 1-minute intervals and smoothed using the Microsoft Excel program with a 10-point moving average.

Data analysis: The data from the frontal chromatography experiments was used to calculate dissociation constants, K_{d}, for the marker and displacer ligands using a previously described approach. The experimental approach is based upon the effect of escalating concentrations of a competitive binding ligand on the retention volume of a marker ligand that is specific for the target receptor. For example, if the NCT receptor is the target, epibatidine (EB) can be used as the marker ligand. Then the association constants of EB, K_{EB}, and the test drug, K_{drug}, as well as the number of the active binding sites of the immobilized NCT receptor, P, can be calculated using Eqn 1 and Eqn 2 set forth below.

(Vₘₐₓ−V)=(1+[EB]K_{EB})(Vₘᵢₙ[P]K_{EB})⁻¹+(1+[EB]K_{EB})²(Vₘᵢₙ[P]K_{EB}K_{drug})⁻¹[drug]⁻¹ (Eqn 1)

(V−Vₘᵢₙ)⁻¹=(Vₘᵢₙ[P]K_{EB})⁻¹+(Vₘᵢₙ[P])⁻¹[EB] (Eqn 2)

In the above equations, V is the retention volume of EB; Vₘₐₓ is the retention volume of EB at low concentration (60 pM) and in the absence of drugs; and Vₘᵢₙ is the retention volume of EB when the specific interaction is completely suppressed. The value of Vₘᵢₙ can be determined by running (³H)-EB in a series of concentration of drugs and plotting 1/(Vₘₐₓ−V) versus 1/(drug) extrapolating to infinite (drug). From the above plot and a plot of 1/(V−Vₘᵢₙ) versus [EB], dissociation constant values, K_{d}, for (³H)-EB and the drugs can be obtained.

Ligands used in this study: NCT Receptor: (³H)-EB was used as the marker; EB and (−)-NCT were used as displacers. GABA_{A} Receptor: (³H)-FTZ was used as the marker; FTZ and diazepam were used as displacers. NMDA Receptor: (³H)-MK-801 was used as the marker; NMDA was used as a displacer.

Results

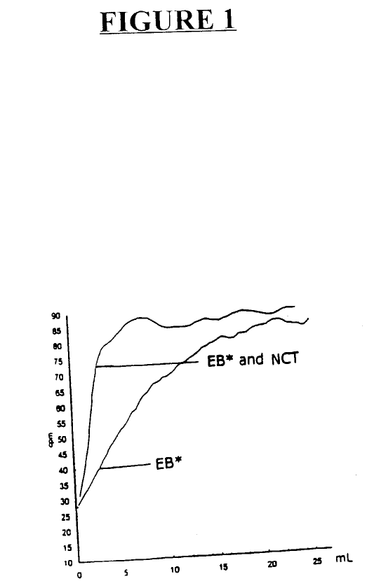

Presence and activity of immobilized nicotinic receptor: In order to determine the presence and activity of the NCT receptor on the MR-SP, the NCT receptor specific ligand (³H)-EB was used as the marker ligand. Chromatographic studies were performed using 60 pM (³H)-EB with varying concentrations of EB (60-450 pM) and (−)-NCT (0.1-1000 nM) as displacer ligands with Tris (50 mM, pH 7.4) as the running buffer. A representative chromatogram depicting the reduction in the breakthrough volume of 60 pM (³H)-EB produced by the addition of 1 μM (1)-NCT to the mobile phase is shown in FIG. 1. In parallel experiments, no specific binding of (³H)-EB was detected on IAM particles.

The results of the chromatographic studies are presented below in Table 1.

TABLE 1

| | K_{d} Frontal (nM) | K_{d} Standard (nM) |
|---|---|---|
| Nicotinic Receptor | | |
| Nicotine | 1.0 | 0.8⁽⁶⁾ |
| Epibatidine | 0.044 | 0.05⁽⁶⁾ |
| GABAA Receptor | | |
| Flunitrazepam | 1.3 | 1.7 |
| Diazepam | 1.0 | 1.3 |
| NMDA Receptor | | |
| NMDA | 1.2/0.6 | 1.5/0.4^{(8,9)} |

The K_{d} values obtained for EB and (−)-NCT from the frontal chromatographic studies are consistent with the values obtained from standard binding studies utilizing membranes prepared from rat brain. Because rat brain tissue contains high concentrations of the α4/β2 NCT receptor (6), the results of the study indicated that the MR-SP contains active NCT receptors, with a high proportion of the α4/β2 NCT receptor subtype.

Presence and Activity of Immobilized GABA_{A} Receptors

In order to determine the presence and activity of the GABA_{A} receptor on the MR-SP, the GABA_{A} receptor specific ligand (³H)-FTZ was used as the marker ligand. Chromatographic studies were then performed using 25 pM (³H)-FTZ with varying concentrations of FTZ and diazepam (0.05-500 nM) as displacer ligands with Tris-HCl buffer (50 mM, pH 7.4) as running buffer. In parallel experiments, no specific binding of (³H)-FTZ was detected on IAM particles.

The results of the chromatographic studies are presented in Table 1. The K_{d} values obtained for FTZ and diazepam from the frontal chromatographic studies are consistent with the values obtained from standard binding studies utilizing membranes prepared from rat brain. Thus, the results of the study indicated that the MR-SP contains active GABA_{A} receptors.

Presence and Activity of Immobilized NMDA Receptors

In order to determine the presence and activity of the NMDA receptor on the MR-SP, the NMDA antagonist, dizocilpine ((³H)-MK-801) was used as the marker ligand. Chromatographic studies were performed using (³H)-MK-801 at a concentration of 2 nM. The chromatographic buffer was Tris-HCl (5 mM, pH 7.4) containing 1 μM L-glutamate and 1 μM glycine. The presence of both amino acids is a prerequisite for the functioning of the NMDA receptor because these two amino acids, which are natural agonists, are required to insure that the receptor is in the right conformation for ligand binding studies.

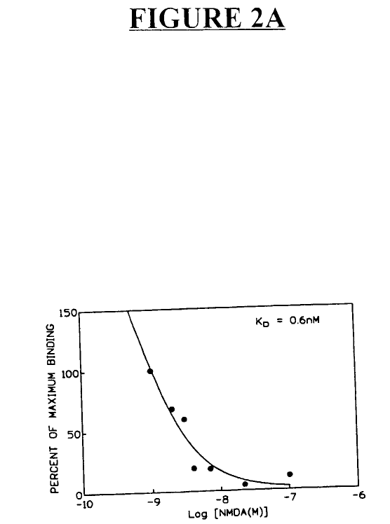

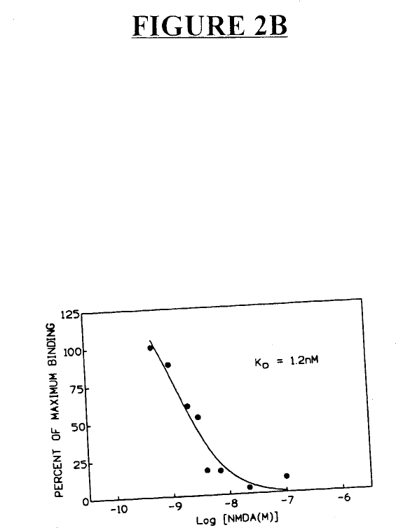

The displacer ligand (NMDA) was added to the chromatographic buffer in increasing concentrations from 1 mM to 2 mM, and the effect on the elution volume of (³H)-MK-801 was determined. NMDA was able to specifically displace (³H)-MK-801, demonstrating that the observed retention was due to binding to the NMDA receptor. In a second experiment, increasing concentrations of unlabeled MK-801 (from 0.5 nM to 200 nM) were added to the elution buffer. The plot of elution volume as a function of NMDA concentration was used to determine the K_{d} of MK-801 for the NMDA receptor. (See FIGS. 2A and 2B). The K_{d} obtained by this method were 0.6 nM and 1.2 nM, which is in agreement with previously published values of 0.4 nM and 1.5-2.0 nM, Table 1.

Interactions between Co-Immobilized GABA_{A} and NCT Receptors

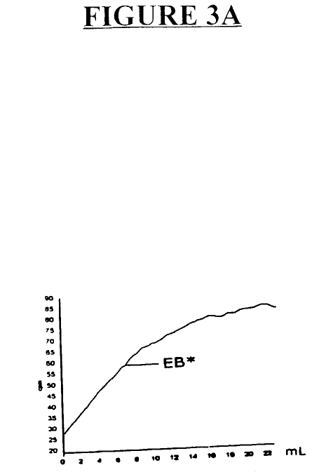

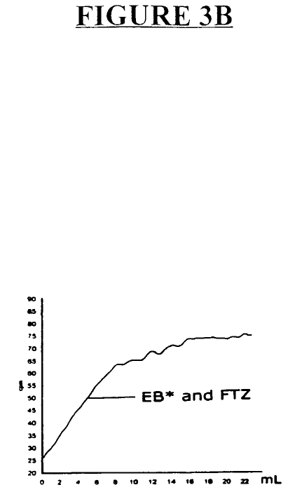

Effect of a GABA_{A} receptor ligand on binding at the NCT receptor: As described above, a series of experiments using the NCT receptor ligand (³H)-EB was conducted and the affinity of EB for the immobilized NCT receptors (K_{d}) was determined to be 0.044 nM. (See Table 1). During these experiments, the (³H)-EB concentration was held constant at 60 pM. At the completion of these studies, the MR-SP was washed with the mobile phase (Tris-HCl buffer (50 mM, pH 7.4)) and 60 pM (³H)-EB was re-injected onto the column. No significant change was seen in the shape or elution volume of the (³H)-EB. The 60 pM (³H)-EB was again injected into the column. However, at this time, the GABA_{A} receptor ligand FTZ has been added to the mobile phase at a 1 μM concentration. Under these experimental conditions, no decrease in the elution volume of (³H)-EB was observed, indicating that the GABA_{A} receptor ligand did not affect the binding of (³H)-EB at the NCT receptor. (See FIGS. 3A and 3B).

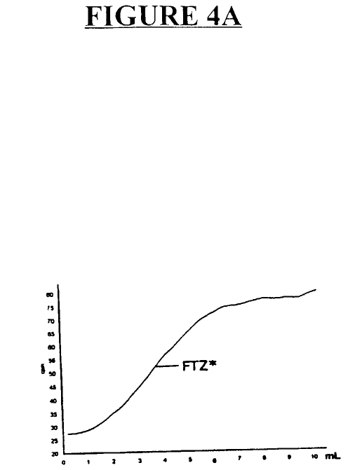

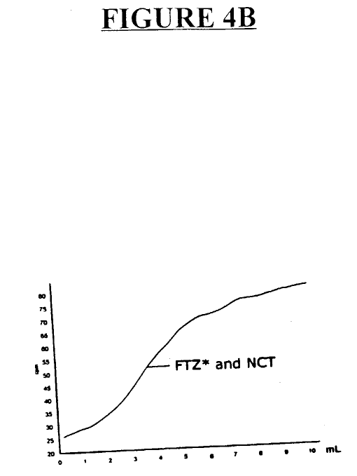

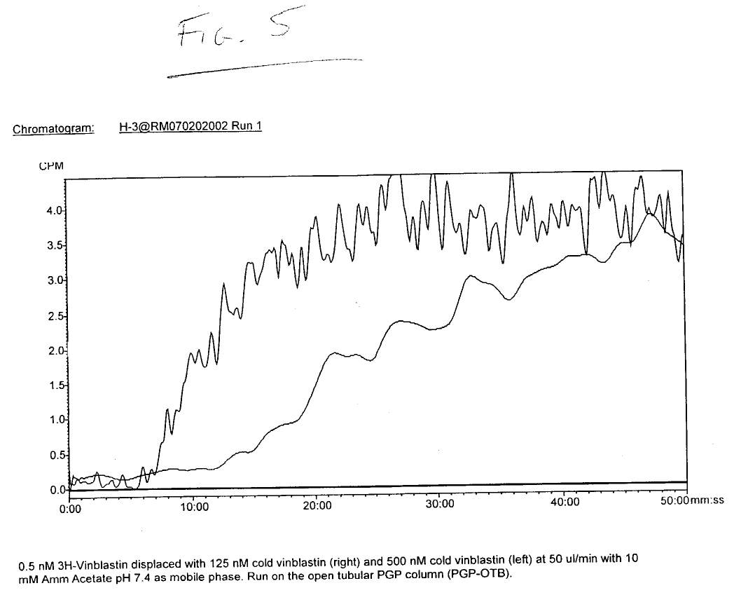

Effect of a NCT receptor ligand on binding at the GABA_{A} receptor: The binding of the GABA_{A} receptor ligand (³H)-FTZ was determined on the MR-SP as described above and the calculated affinity (K_{d}) was determined to be 1.3 nM. During these experiments, the (³H)-FTZ concentration was held constant at 25 pM. At the completion of these studies, the MR-SP was washed with the mobile phase (Tris-HCl buffer (50 mM, pH 7.4)) and 25 pM (³H)-EB was re-injected onto the column. No significant change was seen in the shape or elution volume of the (³H)-FTZ. The 25 pM (³H)-FTZ was again injected onto the column, however, at this time, the NCT receptor ligand (−)-NCT had been added to the mobile phase at a 1 μM concentration. Under these experimental conditions, no decrease in the elution volume of (³H)-FTZ was observed indicating that the NCT receptor ligand did not affect the binding of (³H)-FTZ at the GABA_{A} receptor. (See FIGS. 4A and 4B)

Using methods of the invention, the supports with the receptors, or alternatively with enzymes or transporters, may be exposed to drugs or inhibitors, then to drugs followed by evaluation of the presence of the drug by chromatographic means to determine whether the drug is present on the support.

Using means of the invention, it is also possible to determine whether proposed inhibitors of receptor/toxin interaction will, in fact, prevent that interaction by exposing the support with binding moiety bound thereto to proposed inhibitors, then to the toxin or drug followed by chromatographic evaluation of the support to determine whether the toxin or drug has been prevented from binding to the receptor by the inhibitor under consideration.

Other supports than those exemplified which are HPLC-type supports known in the art may be used. Supports such as hydrogel beads or hydrophilic vertical support systems may be used in the methods of the invention. In the method exemplified, because the method of the invention requires only evaluation of comparative elution volume profiles with the test material being fully eluted at the end of the study, the receptor binding column can be reused repeatedly. Other uses in which one or more of the binding moieties is consumed are also contemplated by the invention.

### Example 2

Another aspect of the invention is a chromatography device that can be used in displacement chromatography, frontal or zonal chromatography and other forms of chromatography. In this alternate construction, there is a chromatography device wherein a binding moiety is covalently bound directly to a support within the column, optionally without the presence or reliance upon a stationary phase for binding moiety immobilization.

Binding moieties immobilized in this way, when characterized as fully developed in vivo, can be proteins that function primarily outside the cell, in the cellular cytosol, or associated with any cellular membrane including the membrane of the cellular wall, the nuclear membrane or the membrane of a cellular organelle.

In one embodiment according to this aspect of the invention, only a single species of binding moiety is immobilized within the chromatography column. This single species of protein, when characterized as fully developed in vivo, can be a single species of protein that functions primarily outside the cell, in the cellular cytosol, or associated with any cellular membrane including the membrane of the cellular wall, the nuclear membrane or the membrane of a cellular organelle.

The species of protein as the covalently bound binding moiety can be a cytosolic protein, a peripheral membrane protein, an integral membrane protein or a transmembrane protein.

In one embodiment, the present invention is also generally concerned with chromatographic systems wherein protein moiety sites may be formed from among different species of one type of one protein moiety. Protein moiety types are differentiated by function and can include functionality classifications based on the cellular activity of the binding moiety such as receptors, cell membrane transporters or channel proteins, and enzymes.

Binding Moiety Immobilized by Covalent Bond

Binding moieties can also be immobilized by a direct covalent bond to a support within the chromatography column. A chromatography device constructed in this manner can optionally contain a stationary phase that may also contain one or more immobilized binding moieties that are not covalently bound to the chromatography column wall. An example demonstrating one embodiment according to this aspect of the invention follows.

PGP-Open Tubular (PGP-OT)

Preparation of open tubular capillary: A 25 cm×100 u ID capillary was cleaned with 0.5 N NaOH running through for 40 minutes at low pressure followed by water for 20 minutes. Then placed in the oven at 95° C. for 30 minutes. Then APTS (90 parts water: 10 parts APTS) was run through the column for 5 minutes at high pressure and placed in the oven for 30 minutes. This was repeated and left overnight. The following morning, a 1% gluteraldehyde solution in PBS was then run through at high pressure for 30 minutes followed by air. Then avidin (10 mg in 4 mL 50 mM PBS) was run through for 3 minutes at high pressure. Then both tips were submerged in the avidin solution overnight at 4° C. It was determined at this stage that the column needs to be washed for 4-6 hours at 50 ul/min with Tris buffer prior to continuing to the next stage. The purpose being to remove any unbound avidin. A frontal study using a 5 mL sample of 0.5 nM ³H-Vinblastin (RM062702002) was carried out to determine if vinblastin had specific binding to avidin. Vinblastins retention time was only 17.30 minutes.

Subsequently, a solution of 14 mM BioX (15 mg of BioX in 1 mL DMSO) was run through the column at high pressure for 10 minutes and left in the solution for 48 hours. A frontal study of 0.5 nM ³H-Vinblastin (5 mL) was carried out on the BiotinX-Avidin labeled OT column to determine if any interactions between vinblastin and biotin could be seen. However, the retention time was only 9.25 minutes. Indicating no interactions with the column.

Preparation of PGP-OT column: 2 mL of homogenization buffer (2 uM Leupeptin; 8 uM Pepstatin A; 2 uM PMSF; 50 mM NaCl in 50 mL of Tris buffer, pH 7.4) was added to 52×10⁶ MDR-1 cells, the solution was homogenized 3×10 s at the setting of 15 on the polytron homogenizer. Then the solution was centrifuged at 450 g for 7 minutes to remove the nuclear proteins. The supernatant was then centrifuged at 35000 rpm for 30 minutes. The pellet was subsequently suspended in 5 mL of solubilization buffer. This was stirred overnight at 150 rpm in the cold room. The following day the solution was centrifuged at 20 000 rpm for 20 minutes. Then the supernatant was run through the prepared capillary (already containing Biotin-X ligand) at high pressure for 4 minutes followed by a 10 minute incubation period, this was repeated twice.

Dialysis of PGP-OT columns: Dialysis tubing was used to cap both ends of the capillary and secured on with copper wiring. The column was then submerged in dialysis buffer (10 mM Tris, 150 mM NaCl, 1.0 mM EDTA, 1 mM Benzamidine) and rotated overnight in the cold room at 110 rpm. The following day the column was run with 10 mM Tris for 3 hours, prior to carrying out experiment on this column, which is referred to as PGP-OTA from this point. The dialysis buffer was replaced on the second day and the procedure was repeated for another 24 hours. Hereby, this column will be referred to as PGP-OTB. After testing each column PGP-OT (RM062602001), PGP-OTA (RM062502001), PGP-OTB (RM062602002), the column with the most reproducible results was fully characterized.

Preparation of PGP(−)-OT column (LCC6-OT): 60×10⁶ LCC-6 cells were homogenized and solubilized and immobilized in the same method as described for the MDR-1 cells. A frontal study of 0.5 nM ³H-Vinblastin was carried out and the retention time was 8.7 minutes (RM072202001).

Results: The K_{d} of Vinblastin for the PGP-OTB column was determined to be 97 nM with an r² value of 0.902. The Bₘₐₓ was determined to be 3 mmoles, indicating that there are 3 mmoles of binding sites for vinblastin on the PGP-OTB column. Various concentrations of verapamil were also shown to displace 0.5 nM ³H-Vinblastin. The PGP-OTB column remained active for 5 weeks.

Direct covalent binding of a protein can be accomplished in a number of ways. For instance, the silica wall is first aminated by treatment with APTS and subsequently any protein can be immobilized with glutaraldehyde or some other condensating reagent, such as an activated ester, to allow for binding and immobilization of a conjugating spacer protein such as avidin or biotin. For instance once avidin is immobilized, this in turn can be biotinylated, followed by binding with a solubilized protein. This can also be carried out using streptavidin or neutravidin as an alternative to avidin. Similarly, alternatives to glutaraldehyde (i.e., glutaric dialdehyde),would be a condensating reagent or an activated ester that can be used to immobilize avidin or other proteins. Examples are EDAC (water-soluble carbodiimide), DCT (dicyclohexylcarbodiimide), and HOBT (4-hydroxy Benzotriazole). Another alternative to immobilizing the binding moiety is simply utilizing a long bifunctional spacer that would first react with the aminated support. This spacer is then in turn reacted with a functional group of a water soluble or membrane protein.

COMBINATION AND ALTERNATIVE COLUMNS—The examples above demonstrate construction of chromatography columns based on distinct architectures. Columns can be prepared combining both architectures such as in a column having one binding moiety covalently bound directly to a support within the column and in combination with a stationary phase, optionally containing one or more binding moieties.

In addition, the amount of any binding moiety can be varied according to the needs of the end-user. The amount of any target binding moiety in a tissue sample will vary depending on the type of tissue used. Endogenous amounts of a target binding moiety in any stationary phase prepared from tissue can vary from 1-2000 fmoles per ml, preferably 10-1000, more preferably 25-500, and still more preferably 50-250 fmoles per ml. These amounts can also be manipulated so any single binding moiety may be present in amounts of above 100 fmoles per ml, preferably above 150 fmoles per ml, more preferably above 200 fmoles per ml, still more preferably above 300 fmoles per ml, even still more preferably above 500 fmoles per ml, 750 fmoles per ml, 1,000 fmoles per ml, 2,000 fmoles per ml, 5,000 fmoles per ml, 10,000 fmoles per ml, 50,000 fmoles per ml and above 100,000 fmoles per ml.

Stationary phase prepared from an expressed cell line may have a much higher molar ratio of the target binding moiety. In addition, extraneous amounts of any target binding moiety can be added in either architecture described above.

The present invention having now been fully described with reference to representative embodiments and details, it will be apparent to one of ordinary skill in the art that changes and modifications can be made thereto without departing from the spirit or scope of the invention as set forth herein.

What is claimed:

1. An artificial membrane support comprising:
(1) a plurality of distinct species of protein as binding moieties non-covalently immobilized thereon, wherein said plurality of immobilized protein binding moieties are immobilized such that the tertiary structure of the protein in each immobilized binding moiety permits specific binding to a molecule that is bound by said protein in said immobilized binding moiety, and
(2) at least one marker molecule associated with both binding moiety protein species.

2. The artificial membrane support according to claim 1, wherein the plurality of distinct binding moieties comprise at least two different species of proteins selected from the group consisting of the genuses of receptors, enzymes and transport proteins.

3. The artificial membrane support according to claim 2, wherein the different species of proteins are selected from one member of the group consisting of the genuses of receptors, enzymes and transport proteins.

4. The artificial membrane support according to claim 2, wherein the different species of protein, are selected from among more than one member of the group consisting of receptors, enzymes and transport proteins.

5. The artificial membrane support according to claim 2, wherein one of the different species of protein is a receptor selected from the group consisting of: acetylcholine receptor, adenosine receptors, adrenergic receptors, adrenomedullin receptor, Ah receptor, amino acid receptors, AMPA (a-Amino-3-hydroxy-5-methyl-4-isoxazole propionic acid) receptor, ANP receptor, androgen receptor, baroreceptor, calcitonin gene related peptide receptor, cannabinoid receptors, chemokine receptors, chemoreceptor, Con A receptors, death receptors, EGF receptor, endothelin receptor, estrogen receptor, Fc receptors, fibroblast growth factor receptor, G-protein-coupled receptor, GABA (gamma aminobutyric acid) receptor, glutamate receptor, glycine receptor, growth factor receptor bound protein 2, glutamate receptor interacting protein, imidazoline receptors, IL-1 receptor associated kinase, insulin receptor substrate-1, immunoreceptor tyrosine-based activation motif, killer cell inhibitory receptor, killer cell immunoglobulin-like receptor, leptin receptor, low density lipoprotein receptor, muscarinic acetylcholine receptor, NCT receptors, α3/β4 NCT receptor-subtype, α4/β2 NCT receptor-subtype, nuclear receptor corepressor, nicotinic acetylcholine receptor, NMDA (N-methyl-D-Aspartate) receptor, nuclear receptor, opioid receptors, peptide neurotransmitter receptor, photoreceptors, peroxisome proliferator-activated receptors, presynaptic receptors, protease-activated receptors, purinergic receptors, receptors for activated C Kinase, receptor tyrosine kinases, scavenger receptors, serpentine receptors, signal recognition particle-receptor, steroid receptor, sulphonylurea receptors; T-cell receptor, TNF receptor, vanilloid receptor-1, thyroid hormone receptors, retinoic acid receptor, progesterone receptor, glucocorticoid receptors, nuclear receptors, ligand gated ion channels, voltage gated ion channels, potassium channel, calcium channel and orphan receptors.

6. The artificial membrane support according to claim 2, wherein one of the different species of protein is a receptor selected from the group consisting of: thyroid hormone receptors, retinoic acid receptor, progesterone receptor, glucocorticoid receptors, nuclear receptors, ligand gated ion channels, voltage gated ion channels, potassium channel, calcium channel and orphan receptors.

7. The artificial membrane support according to claim 2, wherein one of the different species of protein is an enzyme selected from the group consisting of the genuses: (1) oxidoreductases, (2) transferases, (3) proteolytic enzymes, (4) lysases, (5) isomerases, and (6) ligases.

8. The artificial membrane support according to claim 2, wherein one of the different species of protein is an enzyme selected from the group consisting of: abenzyme, angiotensin converting enzyme, apoenzyme, exoenzyme C3, catalytic antibody, coenzymes, coenzyme A, coenzyme M, coenzyme Q, ectoenzyme, endothelin converting enzyme, exoenzyme, holoenzyme, hydrolytic enzymes, interleukin-1 converting enzyme, isoenzymes, lysosomal enzymes, metalloenzyme, modification enzyme, N-acetylglucos-aminyltransferase V, pro-enzymes, Q enzyme, restriction endonucleases, restriction enzymes, coenzyme Q, and orphan enzymes.

9. The artificial membrane support according to claim 2, wherein one of the different species of protein is a transport protein selected from the group consisting of: P-glycoprotein, A-channel, calcium channel, channel-forming ionophore, chloride channel, delayed-rectifier channels, gated ion channel, G-protein-gated inward rectifying potassium channels, ion channels, L-type channels, ligand-gated ion channels, M-channels, N-type channels, P-type channels, potassium channel, Q-type channels, R-type channels, sodium channel, T-type channels, voltage-gated ion channel, voltage-sensitive calcium channels, and orphan transport proteins.

10. A chromatography device comprising the artificial membrane support of claim 1, wherein said artificial membrane support is contained in a liquid flow system.

11. A chromatography device comprising the artificial membrane support of claim 1, wherein said artificial membrane support is produced by the following steps:
(i) obtaining an immobilized artificial membrane (IAM) liquid chromatographic (LC) stationary phase comprising a phospholipid monolayer; and
(ii) contacting said IAM LC stationary phase with a plurality of species of solubilized distinct protein binding moieties under conditions wherein said plurality of immobilized protein binding moieties are non-covalently immobilized such that the tertiary structure of the protein in each immobilized binding moiety permits specific binding to a molecule that is bound by the protein.

12. A method of using a chromatography device according to claim 10, comprising exposing the artificial membrane support to a liquid flow system.

13. The method of use according to claim 12, wherein said use is to investigate single or multiple interactions between at least one species of molecule and a plurality of species of protein binding moieties.

14. The method of use according to claim 12, wherein said use is to identify new drug candidates.

15. The method of use according to claim 12, wherein said use is to isolate a compound from a complex biological matrix.

16. A method of using a chromatography device comprising an artificial membrane support comprising a plurality of species of protein binding moieties non-covalently immobilized thereon, wherein said plurality of species of immobilized protein binding moieties are immobilized such that the tertiary structure of the protein in each immobilized binding moiety permits specific binding to a molecule that is bound by said protein in said immobilized protein binding moiety, said method comprising: exposing said artificial membrane support to a liquid flow system.

17. The method of use according to claim 16, wherein said use is to investigate single or multiple interactions between at least one species of molecule and a plurality of of species of protein binding moieties.

18. The method of use according to claim 16, wherein said use is to identify new drug candidates.

19. The method of use according to claim 16, wherein said use is to isolate a compound from a complex biological matrix.

20. A support comprising
(1) at least one species of protein binding moiety immobilized through a covalent bond with the support surface to form an immobilized protein binding moiety, wherein said species of immobilized protein binding moiety is immobilized such that the protein in the immobilized binding moiety permits specific binding to a molecule that is bound by said protein in said immobilized protein binding moiety, and
(2) at least one marker molecule associated with the protein binding moiety species.

21. The support according to claim 20, wherein said binding moiety protein is a cytosolic protein.

22. The support according to claim 20, wherein said binding moiety protein is a membrane protein.

23. The support according to claim 20, wherein said binding moiety protein is a peripheral membrane protein.

24. The support according to claim 20, wherein said binding moiety protein is a transmembrane membrane protein.

25. The support according to claim 20, wherein said binding moiety comprises a species of protein selected from one member of the group consisting of the genuses of receptors, enzymes and transport proteins.

26. The support according to claim 20, wherein said binding moiety protein is a receptor selected from the group consisting of: acetylcholine receptor, adenosine receptors, adrenergic receptors, adrenomedullin receptor, Ah receptor, amino acid receptors, AMPA (α-Amino-3-hydroxy-5-methyl-4-isoxazole propionic acid) receptor, ANP receptor, androgen receptor, baroreceptor, calcitonin gene related peptide receptor, cannabinoid receptors, chemokine receptors, chemoreceptor, Con A receptors, death receptors, EGF receptor, endothelin receptor, estrogen receptor, Fe receptors, fibroblast growth factor receptor, G-protein-coupled receptor, GABA (gamma aminobutyric acid) receptor, glutamate receptor, glycine receptor, growth factor receptor bound protein 2, glutamate receptor interacting protein, imidazoline receptors, IL-1 receptor associated kinase, insulin receptor substrate-1, immunoreceptor tyrosine-based activation motif, killer cell inhibitory receptor, killer cell immunoglobulin-like receptor, leptin receptor, low density lipoprotein receptor, muscarinic acetylcholine receptor, NCT receptors, α3/β4 NCT receptor-subtype, α4/β2 NCT receptor-subtype, nuclear receptor corepressor, nicotinic acetylcholine receptor, NMDA (N-methyl-D-Aspartate) receptor, nuclear receptor, opioid receptors, peptide neurotransmitter receptor, photoreceptors, peroxisome proliferator-activated receptors, presynaptic receptors, protease-activated receptors, purinergic receptors, receptors for activated C Kinase, receptor tyrosine kinases, scavenger receptors, serpentine receptors, signal recognition particle-receptor, steroid receptor, sulphonylurea receptors; T-cell receptor, TNF receptor, vanilloid receptor-1, thyroid hormone receptors, retinoic acid receptor, progesterone receptor, glucocorticoid receptors, nuclear receptors, ligand gated ion channels, voltage gated ion channels, potassium channel, calcium channel and orphan receptors.

27. The support according to claim 20, wherein said binding moiety protein is a receptor selected from the group consisting of: thyroid hormone receptors, retinoic acid receptor, progesterone receptor, glucocorticoid receptors, nuclear receptors, ligand gated ion channels, voltage gated ion channels, potassium channel, calcium channel and orphan receptors.

28. The support according to claim 20, wherein said binding moiety protein is an enzyme selected from the group consisting of the genuses: (1) oxidoreductases, (2) transferases, (3) proteolytic enzymes, (4) lysases, (5) isomerases, and (6) ligases.

29. The support according to claim 20, wherein said binding moiety protein is an enzyme selected from the group consisting: abenzyme, angiotensin converting enzyme, apoenzyme, exoenzyme C3, catalytic antibody, coenzymes, coenzyme A, coenzyme M, coenzyme Q, ectoenzyme, endothelin converting enzyme, exoenzyme, holoenzyme, hydrolytic enzymes, interleukin-1 converting enzyme, isoenzymes, lysosomal enzymes, metalloenzyme, modification enzyme, N-acetylglucos-aminyltransferase V, pro-enzymes, Q enzyme, restriction endonucleases, restriction enzymes, coenzyme Q, and orphan enzymes.

30. The support according to claim 20, wherein said binding moiety protein is a transport protein selected from the group consisting of: P-glycoprotein, A-channel, calcium channel, channel-forming ionophore, chloride channel, delayed-rectifier channels, gated ion channel, G-protein-gated inward rectifying potassium channels, ion channels, L-type channels, ligand-gated ion channels, M-channels, N-type channels, P-type channels, potassium channel, Q-type channels, R-type channels, sodium channel, T-type channels, voltage-gated ion channel, voltage-sensitive calcium channels, and orphan transport proteins.

31. The support according to claim 20, and further comprising at least one additional binding moiety that comprises a distinct species of protein from that in said at least one binding moiety protein immobilized through a covalent bond with the support wall.

32. A method of using a chromatography device comprising a support comprising at least one protein binding moiety immobilized through a covalent bond with the support surface to form an immobilized protein binding moiety, wherein said immobilized protein binding moiety is immobilized such that the protein in the immobilized binding moiety permits specific binding to a molecule that is bound by said protein in said immobilized protein binding moiety, said method comprising:
exposing said support to a liquid flow system containing a marker molecule associated with the protein binding moiety species.

33. The method of use according to claim 32, wherein said use is to investigate single or multiple interactions between at least one species of molecule and at least one species of protein binding moiety.

34. The method of use according to claim 32, wherein said use is to identify new drug candidates.

35. The method of use according to claim 32, wherein said use is to isolate a compound from a complex biological matrix.

36. A method for performing drug discovery comprising using a chromatography device having an artificial membrane support comprising a plurality of distinct binding moieties non-covalently immobilized thereon, wherein said plurality of immobilized binding moieties are immobilized such that the tertiary structure of the protein in each immobilized binding moiety permits specific binding to a molecule that is bound by said protein in said immobilized binding moiety, comprising:
exposing said support to a liquid flow system containing a marker molecule associated with the protein binding moiety species in a process of lead optimization.

37. The method according to claim 36, wherein the lead optimization process involves gathering data toward analyzing the adsorption, distribution, metabolism, excretion, or the toxicological effect of a molecule.

38. The method according to claim 36, wherein the plurality of distinct binding moieties comprise different species of proteins selected from the group consisting of the genuses of receptors, enzymes, transport proteins or other binding proteins.

39. A method for performing drug discovery comprising using a chromatography device having a support comprising at least one binding moiety immobilized through a covalent bond with the support surface to form an immobilized binding moiety, wherein said immobilized binding moiety is immobilized such that a protein in the immobilized binding moiety permits specific binding to a molecule that is bound by said protein in said immobilized binding moiety, comprising:
exposing said support to a liquid flow system containing a marker molecule associated with the protein binding moiety species in a process of lead optimization.

40. The method according to claim 39, wherein the lead optimization process involves gathering data toward analyzing the adsorption, distribution, metabolism, excretion, or the toxicological effect of a molecule.

41. The method according to claim 39, wherein the at least one binding moiety immobilized through a covalent bond with the support surface to form an immobilized binding moiety comprises a species of protein selected from the group consisting of the genuses of receptors, enzymes, transport proteins or other binding proteins.

42. A method of making an artificial membrane support comprising a plurality of distinct binding moieties non-covalently immobilized thereon, wherein said plurality of immobilized binding moieties are immobilized such that the tertiary structure of the protein in each immobilized binding moiety permits specific binding to at least one molecule that is bound by a protein in said plurality of immobilized binding moieties, comprising:
forming a stationary phase containing a plurality of species of protein having a known binding affinity for a least one marker molecule and combining with a marker molecule associated with the protein binding moiety species.

43. A method of making a support comprising at least one binding moiety immobilized through a covalent bond with the support surface to form an immobilized binding moiety, wherein said immobilized binding moiety is immobilized such that a protein in the immobilized binding moiety permits specific binding to a molecule that is bound by said protein in said immobilized binding moiety:
forming a covalent bond linking a protein having a known binding affinity for at least one marker molecule with a surface of the support and combining a marker molecule associated with the protein binding moiety species.
